(19) United States
(12) Patent Application Publication
(10) Pub. No.: US 2014/0114346 A1
(43) Pub. Date: Apr. 24, 2014

(54) Transapical Entry Point Closure Device

(71) Applicant: MEDTRONIC, INC., Minneapolis, MN (US)

(72) Inventor: Gerry McCaffrey, Galway (IE)

(73) Assignee: MEDTRONIC, INC., Minneapolis, MN (US)

(21) Appl. No.: 13/658,296

(22) Filed: Oct. 23, 2012

(51) Int. Cl. A61B 17/08

(52) U.S. Cl. 606/213

(57) ABSTRACT

The medical devices and methods disclosed herein can be used to close a transapical entry point after a medical procedure, or various other anatomical apertures. The medical devices can include a first disc and a second disc, which can be compressed within a catheter, having a distal surface and a proximal surface. A shaft can be attached to, and extend from, the first disc. The second disc can have a hole sized such that the shaft can fit through the hole. In certain embodiments, the proximal surface of the first disc and the distal surface of the second disc can include a plurality of tissue engagement elements, such as teeth. In certain embodiments, the shaft can include a plurality of locking notches such that the second disc can be advanced only in the distal direction over the locking notches.

### BACKGROUND

1. Field

The present disclosure relates to medical devices and methods for closing anatomical apertures. More specifically, the present disclosure relates to medical devices for closing entry points used during transapical procedures and methods of implanting such medical devices. However, it is understood that the medical devices and methods disclosed herein can be used in other types of procedures and at other locations in the body for closing various anatomical apertures.

2. Background

There are numerous surgical procedures for accessing and operating on various parts of the heart. One method of accessing the interior of the heart uses a transapical approach. In transapical procedures, the apex of the heart can be accessed, such as by mini-sternotomy or thoracotomy. An incision can be made in the apex of the heart to provide an entry point for inserting, for example, a catheter to be used during the medical procedure.

Upon completion of the medical procedure, the entry point at the apex must be closed. One method of closing such an entry point is by sewing the entry point together with sutures. However, in a procedure performed on the beating heart, as can be the case in transapical procedures, there are certain risks associated with using sutures to pierce through the moving heart tissue. The medical devices and methods disclosed herein can provide fast and effective means for closing an entry point after performing a transapical procedure without the use of sutures.

### BRIEF SUMMARY

The present disclosure relates to medical devices for closing anatomical apertures, such as, but not limited to, entry points used during transapical heart procedures. It is understood that the medical devices and methods disclosed herein can also be used in other procedures and in other locations of the body, such as, but not limited to, closing an opening in the septal wall.

The medical devices disclosed herein can be used to close a transapical entry point after a medical procedure. The use of a locking, self-closing device such as those illustrated herein can also be used for closing other anatomical apertures. In certain embodiments, a first disc can fold downward to be inserted into a delivery tool. It can expand to its original form and engage an interior tissue surface once inside the body lumen and deployed from the delivery tool. A second disc can be pushed up a shaft until it contacts the exterior tissue surface. The second disc can then be deployed from the delivery tool. The excess portion of the shaft can then be removed so that the shaft is flush with the proximal surface of the second disc.

The medical devices for closing an anatomical aperture can include a first disc and a second disc, each of which can be configured to be compressed within a catheter, having a distal surface and a proximal surface. Throughout the disclosure, the first disc may also be referred to as the “plug” and the second disc may also be referred to as the “head”. A shaft can be attached to, and extend from, the first disc. The second disc can have a hole sized such that the shaft can fit through the hole. In certain embodiments, the hole can be centered about a central axis of the second disc. In certain embodiments, the proximal surface of the first disc and the distal surface of the second disc can include a plurality of tissue engagement elements. The tissue engagement elements can be, for example, a plurality of teeth. In certain embodiments, an exterior surface of the shaft and an interior surface of the hole in the second disc can be threaded, such that the second disc can be advanced along the shaft by rotating the second disc. In certain embodiments, the shaft can include a plurality of locking notches such that the second disc can be advanced only in the distal direction over the locking notches.

Delivery systems for delivering the medical devices described herein are also disclosed. The delivery systems can include a delivery tool for delivering and deploying the medical device. The delivery tool can include a delivery shaft as well as an advancement member configured to advance the second disc distally along the shaft attached to the first disc. The delivery tool can also include a decoupling member, configured to remove a proximal portion of the shaft after the second disc is advanced along the shaft. In certain embodiments, the decoupling member can include a tube having an interior lumen sized to fit about the shaft, and at least one blade located at a distal end of the tube, configured to close about the shaft and cut a portion of the shaft located proximally from the second disc after it is advanced along the shaft. In certain embodiments, the advancement member and the decoupling member can be a single tool.

Methods of delivering a medical device and closing an anatomical aperture are also disclosed. Delivery systems carrying medical devices such as the ones disclosed herein can be inserted into a body lumen and advanced to a deployment location. An outer shaft of the delivery tool can be retracted to deploy the first disc, allowing it to expand to an unconstrained deployment configuration. The delivery system can then be retracted to engage the first disc with an interior tissue wall. The second disc can be advanced along the shaft, for example, by pushing it with an advancement member, to engage the second disc with an exterior tissue wall. The outer shaft can then be retracted to deploy the second disc, allowing it to expand to an unconstrained deployment configuration. In certain embodiments, a decoupling member can be advanced within the delivery tool to remove a portion of the shaft located proximally from the second disc.

### BRIEF DESCRIPTION OF THE DRAWINGS/FIGURES

FIG. 1A illustrates an oblique view of the medical device, according to an embodiment.

FIG. 1B illustrates both a front view of the medical device and a bottom view of the first disc, according to an embodiment.

FIG. 2A illustrates the first disc in a folded configuration attached to the shaft, according to an embodiment.

FIG. 2B illustrates the second disc in a compressed and expanded state, according to an embodiment.

FIG. 2C illustrates both discs attached to the shaft, according to an embodiment.

FIG. 2D illustrates a portion of the shaft removed after the second disc is advanced along the shaft, according to an embodiment.

FIG. 3A illustrates a delivery tool, carrying the first disc attached to the shaft, advanced through an anatomical aperture, according to an embodiment.

FIG. 3B illustrates the delivery tool retracted to deploy the first disc, according to an embodiment.

FIGS. 3C and 3D illustrate the second disc being pushed along the shaft by an advancement member, according to an embodiment.

FIGS. 3E and 3F illustrate a decoupling member removing a portion of the shaft, according to an embodiment.

FIG. 3G illustrates retraction of the delivery tool, leaving the medical device in place, according to an embodiment.

### DETAILED DESCRIPTION

While the disclosure refers to illustrative embodiments for particular applications, it should be understood that the disclosure is not limited thereto. Modifications can be made to the embodiments described herein without departing from the spirit and scope of the present disclosure. Those skilled in the art with access to this disclosure will recognize additional modifications, applications, and embodiments within the scope of this disclosure and additional fields in which the disclosed examples could be applied. Therefore, the following detailed description is not meant to be limiting. Further, it is understood that the systems and methods described below can be implemented in many different embodiments of hardware. Any actual hardware described is not meant to be limiting. The operation and behavior of the systems and methods presented are described with the understanding that modifications and variations of the embodiments are possible given the level of detail presented.

References to “one embodiment,” “an embodiment,” “in certain embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

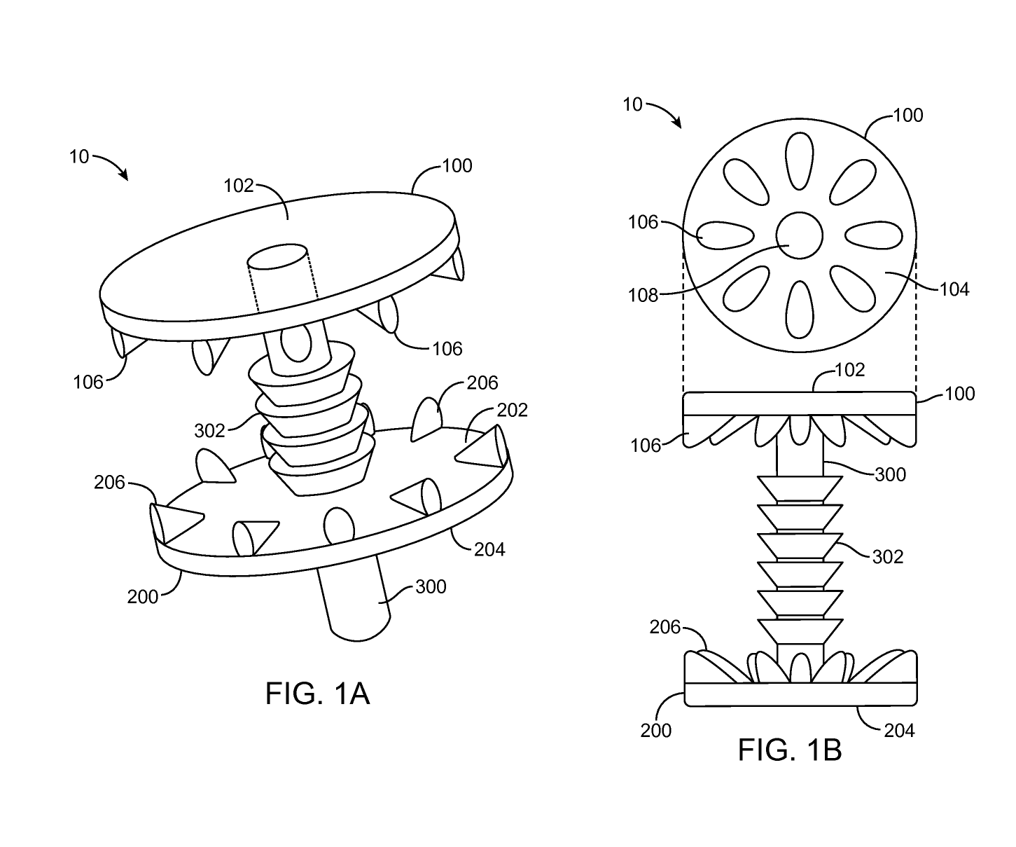

FIGS. 1A and 1B illustrate various views of medical device 10, according to an embodiment. In certain embodiments, medical device 10 can include a first disc, plug 100, having distal surface 102 and proximal surface 104. In certain embodiments, plug 100 can include a plurality of tissue engagement elements, such as, but not limited to, teeth 106. Similar to plug 100, the second disc, head 200, can have distal surface 202 and proximal surface 204, as well as tissue engagement elements, such as teeth 206. Generally, plug 100 and head 200 can be flat, cylindrical shapes, although they can be any shape, for example, square, ellipsoid or hexagonal.

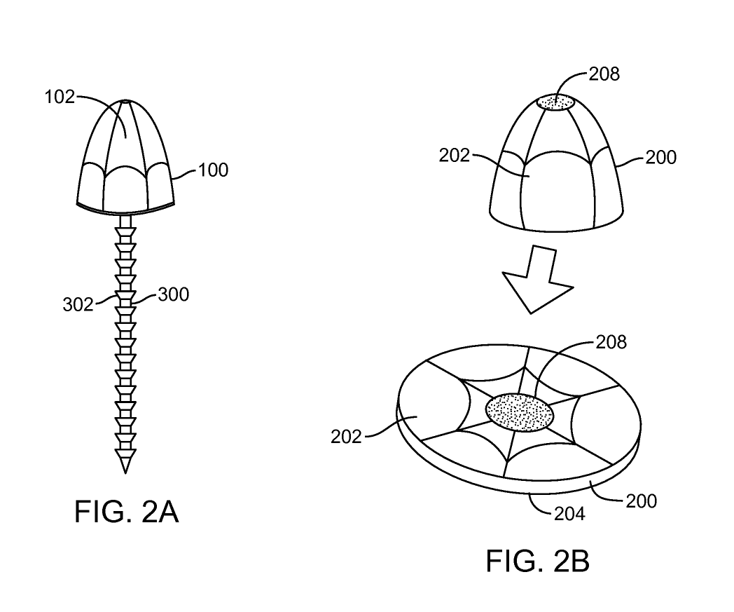

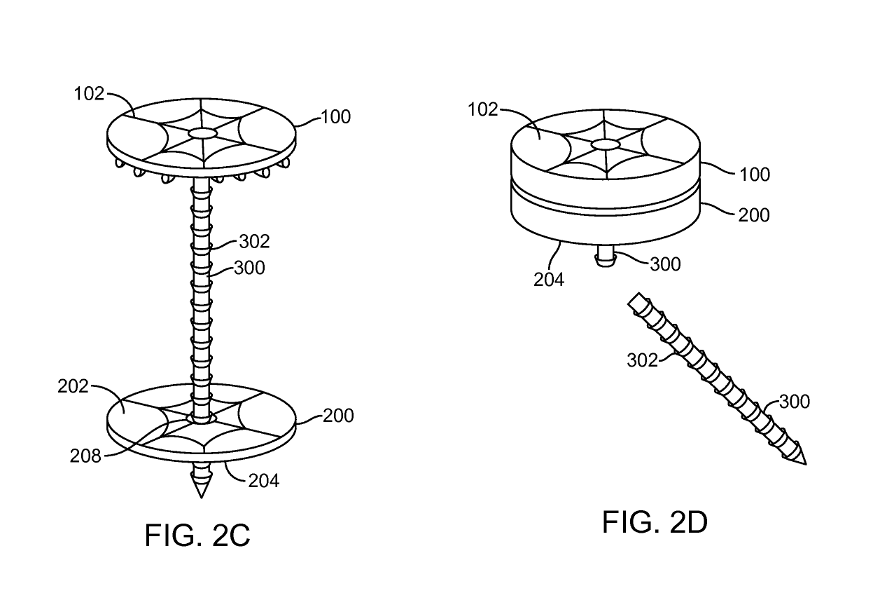

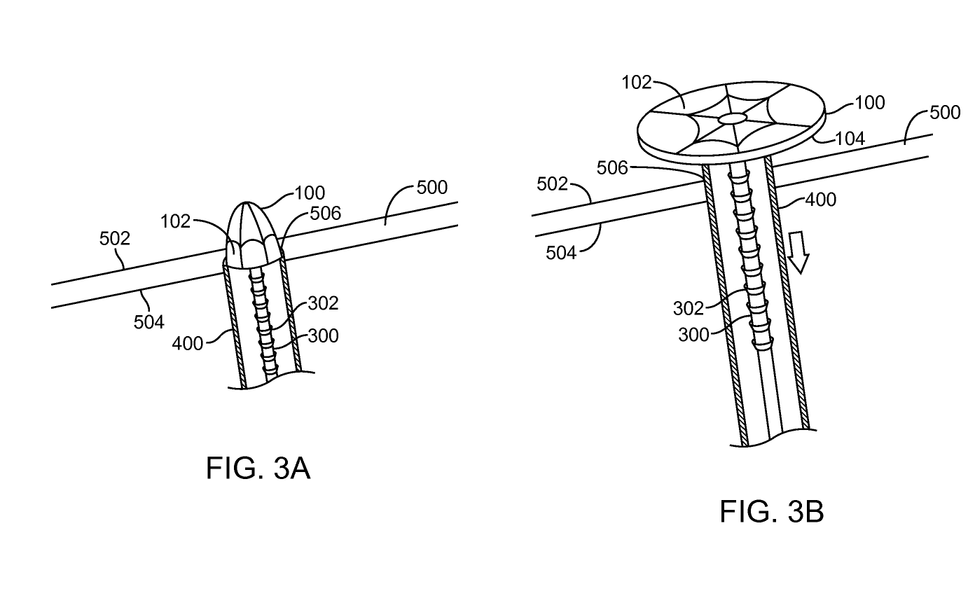

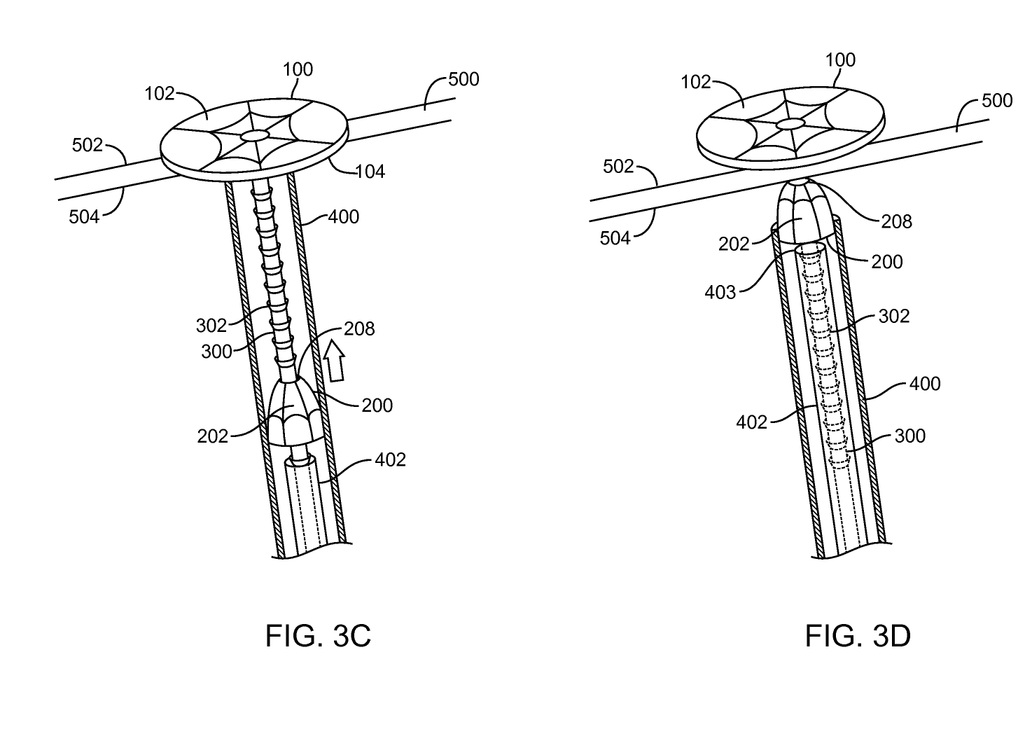

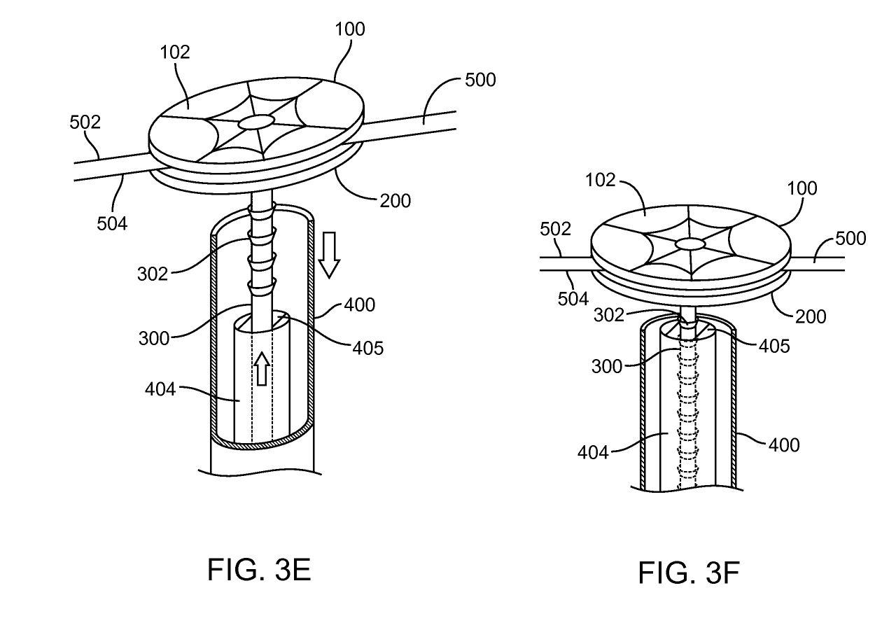

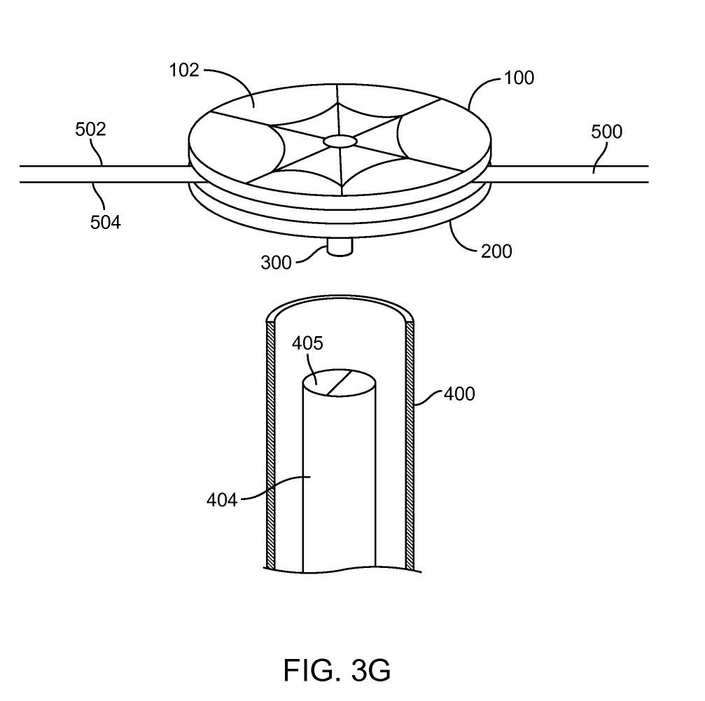

Generally, plug 100 and head 200 can be folded in such a way to create a rounded profile, which can potentially act as a guide tip, as illustrated in FIGS. 2A and 3A. In certain embodiments, plug 100 and head 200 can be made of a shape-memory alloy, such as, but not limited to, nitinol. In certain embodiments, plug 100 and head 200 can include a nitinol frame encapsulated in a polymer. Plug 100 and head 200 can also be made from any other material capable of being folded into a rounded profile to fit within a delivery shaft and capable of re-expanding once deployed from the delivery shaft. In certain embodiments, plug 100 and head 200 can be biased to a preset shape, such that they return to the preset shape after being deployed from a delivery shaft. In certain embodiments, plug 100 and head 200 can be made from a rigid material, and delivered through the delivery shaft in their expanded configuration.

In certain embodiments, tissue engagement elements can be included on proximal surface 104 of plug 100 and distal surface 202 of head 200. In certain embodiments, the tissue engagement elements can be teeth 106 and 206. It is understood that reference to teeth 106 and 206 includes any other element configured to engage tissue, such as hooks, barbs or a textured surface. In certain embodiments, teeth 106 and 206 can be spaced circumferentially about proximal surface 104 of plug 100 and distal surface 202 of head 200, respectively. In certain embodiments, teeth 106 and 206 can be tapered from an outer circumference toward a central axis of plug 100 and head 200.

Shaft 300 can be connected to plug 100, for example, at attachment point 108 shown in FIG. 1B. In certain embodiments, shaft 300 can be attached to proximal surface 104 of plug 100. In certain embodiments, shaft 300 can extend through plug 100 and attach to distal surface 102. It is understood that any form of attaching shaft 300 to plug 100 can be used.

Shaft 300 can be made of any suitable material, such as, but not limited to, biocompatible polymers or metals. In certain embodiments, plug 100 and shaft 300 can be formed from a single molded material, such as a polymer. In certain embodiments, shaft 300 can have a threaded exterior surface, which can engage a threaded interior surface of hole 208 of head 200. Thus, by rotating head 200, it can be advanced distally along shaft 300.

In certain embodiments, shaft 300 can include locking notches 302. Locking notches 302 can be, for example, protrusions or grooves along an exterior surface of shaft 300. In certain embodiments, locking notches 302 can be tapered from a distal end to a proximal end, such that head 200 can be advanced only in the distal direction over locking notches 302. A certain amount of force can be necessary to advance head 200 over each locking notch 302 on shaft 300. In certain embodiments, shaft 300 and locking notches 302 can be coated with a biocompatible lubricant to facilitate advancing head 200 along shaft 300. In certain embodiments, a distal circumference of shaft 300 can be greater than a proximal circumference of shaft 300, such that a frictional interference fit can be created as head 200 is advanced distally along shaft 300. After head 200 is advanced along shaft 300, an excess portion of shaft 300 extending proximally from proximal surface 204 of head 200 can be removed, for example, by cutting or snapping shaft 300.

FIG. 2A illustrates plug 100 attached to shaft 300. As illustrated, plug 100 can be folded proximally to create a rounded profile. This rounded profile can allow plug 100 to be inserted into a delivery tool with a diameter smaller than the expanded diameter of plug 100. It can also allow plug 100 to act as a guide tip for the delivery tool as it is advanced through anatomical aperture 506 in tissue 500, as illustrated in FIG. 3A. Similarly, FIG. 2B illustrates head 200 folded to create a rounded profile for advancing head 200 along shaft 300. FIG. 2B also shows head 200 in its expanded state. Hole 208 is shown, which can allow head 200 to be advanced along shaft 300.

FIG. 2C illustrates both plug 100 and head 200 attached to shaft 300. In certain embodiments, head 200 can be advanced along shaft 300 in its expanded state. Generally, however, head 200 can be folded to create a rounded profile to decrease the necessary delivery shaft diameter. In certain embodiments, plug 100 and head 200 can both be attached to shaft 300 when loaded into the delivery shaft. In certain embodiments, head 200 can be advanced within the delivery shaft and along shaft 300 after plug 100 is deployed. Shaft 300 can be engaged with a proximal portion of the delivery tool so that it does not move distally as head 200 is advanced distally along shaft 300, such as by securing it with a handle at the proximal end of the delivery tool. As shown in FIG. 2D, once head 200 is advanced distally along shaft 300, a proximal portion of shaft 300 can be removed. Part of shaft 300 can be left protruding from head 200, or the proximal portion shaft 300 can be removed such that it is flush with proximal surface 204 of head 200.

FIGS. 3A through 3G illustrate the delivery and implantation sequence for medical device 10. In certain embodiments, a delivery tool can be used to deliver medical device 10. In certain embodiments, the delivery tool can include delivery shaft 400, advancement member 402 and decoupling member 404. In certain embodiments, delivery shaft 400 can be a catheter with an interior lumen within which medical device 10 can be advanced. In certain embodiments, the delivery tool can include a rigid shaft and resemble a rivet gun. Delivery shaft 400 can be made from any biocompatible material, for example, plastic or metal.

FIG. 3A illustrates delivery shaft 400 containing plug 100 in a folded configuration and attached to shaft 300, as delivery shaft 400 is advanced through anatomical aperture 506 in tissue 500. Plug 100 can act as a guide tip for delivery shaft 400 as it is advanced through anatomical aperture 506 in tissue 500. Plug 100 can be deployed by advancing plug 100 out of a hole located at a distal end of delivery shaft 400. In certain embodiments, delivery shaft 400 can be retracted to deploy plug 100. As shown in FIG. 3B, deployment of plug 100 can allow plug 100 to expand from its folded delivery configuration to an expanded deployment configuration. The expansion of plug 100 can be facilitated, for example, by the nature of the material from which plug 100 is made, such as a shape-memory alloy. As indicated by the arrow in FIG. 3B, the delivery tool, along with shaft 300 and plug 100, can then be pulled in the proximal direction, such that plug 100 contacts interior wall 502 of tissue 500. This can allow teeth 106 to engage interior wall 502 of tissue 500. In certain embodiments, a tether can be attached to shaft 300 and pulled in the proximal direction to maintain contact between plug 100 and interior wall 502 while head 200 is advanced along shaft 300. The tether can later be removed along with the excess proximal portion of shaft 300.

FIGS. 3C and 3D illustrate head 200 being advanced along shaft 300 by advancement member 402. In certain embodiments, advancement member 402 can be a tube with lumen 403. Lumen 403 can be sized to fit about shaft 300, in order to advance head 200 along shaft 300. As illustrated by the arrow in FIG. 3C, advancement member 402 can push head 200 in the distal direction along shaft 300. The tapered nature of locking notches 302 can allow head 200 to slide along shaft 300 in the distal direction, but prevent head 200 from sliding back along shaft 300 in the proximal direction. Head 200 can be advanced over each locking notch 302 until head 200 contacts exterior wall 504 of tissue 500, as illustrated in FIG. 3D.

As illustrated by the arrow outside of delivery shaft 400 in FIG. 3E, the delivery tool, including delivery shaft 400, can be retracted in the proximal direction to deploy head 200 and allow head 200 to expand from its delivery configuration within delivery shaft 400 to its expanded deployment configuration. Tissue 500 can thus be sandwiched between proximal surface 104 of plug 100 and distal surface 202 of head 200. Teeth 206 located on distal surface 202 of head 200 can engage exterior wall 504 of tissue 500. In certain embodiments, shaft 300 can be rotated to further engage teeth 106 and 206 with tissue 500.

Advancement member 402 can be retracted, and as illustrated by the arrow in FIG. 3E, decoupling member 404 can be advanced within delivery shaft 400. Decoupling member 404 can have an interior lumen such it can pass around and about shaft 300. In certain embodiments, decoupling member 404 can include one or more blades 405, which can open, as illustrated in FIG. 3F, to allow shaft 300 to pass within decoupling member 404. Blades 405 can then be closed to cut shaft 300. In certain embodiments, decoupling member 404 can include other means of removing the excess portion of shaft 300 located proximally from head 200. For example, decoupling member 404 can include gripping members which can be used to grip and snap shaft 300. Shaft 300 can include perforations or break away locations such that the removal of the excess portion of shaft 300 leaves a smooth surface. In certain embodiments, advancement member 402 and decoupling member 404 can be configured as a single tool, such that the tool can advance head 200 along shaft 300 and then remove the excess portion of shaft 300 located proximally from head 200.

As shown in FIG. 3G, after decoupling member 404 removes the excess portion of shaft 300, medical device 10 can be left in place, closing anatomical aperture 506 in tissue 500. The delivery tool, including delivery shaft 400 and decoupling member 404, can then be removed from the body.

Methods of delivering a medical device for closing an anatomical aperture are also disclosed. References to the figures are meant by way of example, and are not meant to be limiting. After completion of a surgical procedure, such as a transapical procedure, medical device 10 can be loaded into a delivery tool. Delivery shaft 400 of the delivery tool can be advanced to a deployment location. Plug 100 and head 200 can be preloaded onto shaft 300 and into delivery shaft 400, or advanced within delivery shaft 400 after it is positioned at the deployment location. In certain embodiments, plug 100 and head 200 can be advanced within delivery shaft 400 in a folded delivery configuration.

Delivery shaft 400 can be retracted in the proximal direction or plug 100 can be advanced in the distal direction to deploy plug 100, allowing plug 100 to expand to an unconstrained deployment configuration. The delivery tool and plug 100 can then be retracted to engage plug 100 with interior wall 502 of tissue 500. Head 200 can then be advanced along shaft 300 to engage head 200 with exterior wall 504 of tissue 500. Delivery shaft 400 can then be retracted in the proximal direction to deploy head 200, allowing head 200 to expand to its unconstrained deployment configuration. Once medical device 10 is deployed, delivery shaft 400 can be removed from the body.

In certain embodiments, head 200 can be advanced along shaft 300 by pushing head 200 with advancement member 402. A certain amount of force can be required to push head 200 over locking notches 302. Head 200 can be pushed over each locking notch 302, one at a time. In certain embodiments, an exterior surface of shaft 300 and an interior surface of hole 208 in head 200 can be threaded. Advancement member 402 can thus be used to rotate head 200 in order to advance head 200 along the threaded surface of shaft 300. In certain embodiments, advancement member 402 can engage a groove or fixation member located on proximal surface 204 of head 200 to facilitate advancing head 200 along shaft 300. Once head 200 is advanced to tissue 500, advancement member 402 can be disengaged from head 200 and removed from delivery shaft 400.

In certain embodiments, decoupling member 404 can be advanced within delivery shaft 400 to remove an excess portion of shaft 300 located proximally from head 200. Decoupling member 404 can have an interior lumen and distal opening so that it can advance around shaft 300. In certain embodiments, decoupling member 404 can include one or more blades 405, which can be used to cut shaft 300. In certain embodiments, decoupling member 404 can be used to grip shaft 300 and snap off the excess portion of shaft 300. In certain embodiments, advancement member 402 and decoupling member 404 can be combined into a single tool which can push head 200 along shaft 300 and then remove the excess portion of shaft 300.

The foregoing description has been presented for purposes of illustration and description. It is not intended to be exhaustive or to limit the precise embodiments disclosed. Other modifications and variations may be possible in light of the above teachings. The embodiments and examples were chosen and described in order to best explain the principles of the embodiments and their practical application, and to thereby enable others skilled in the art to best utilize the various embodiments with modifications as are suited to the particular use contemplated. By applying knowledge within the skill of the art, others can readily modify and/or adapt for various applications such specific embodiments, without undue experimentation, without departing from the general concept. Therefore, such adaptations and modifications are intended to be within the meaning and range of equivalents of the disclosed embodiments, based on the teaching and guidance presented herein.

What is claimed is:

1. A medical device for closing an anatomical aperture comprising:
a first disc, configured to be compressed within a delivery shaft, comprising a distal surface and a proximal surface;
a shaft attached to the first disc and extending from the proximal surface of the first disc; and
a second disc, configured to be compressed within the delivery shaft, comprising a distal surface and a proximal surface, the second disc having a hole, wherein the hole is sized such that the shaft can fit through the hole.

2. The medical device of claim 1, wherein the proximal surface of the first disc and the distal surface of the second disc further comprise a plurality of tissue engagement elements.

3. The medical device of claim 2, wherein the tissue engagement elements comprise a plurality of teeth.

4. The medical device of claim 3, wherein the teeth are spaced circumferentially about the discs.

5. The medical device of claim 4, wherein the teeth are tapered from an outer circumference of the discs toward a central axis of the discs.

6. The medical device of claim 1, wherein a distal circumference of the shaft is greater than a proximal circumference of the shaft.

7. The medical device of claim 1, wherein an exterior surface of the shaft is threaded, and wherein an interior surface of the hole of the second disc is threaded.

8. The medical device of claim 1, wherein the shaft further comprises a plurality of locking notches, wherein the locking notches are tapered from a distal end to a proximal end, such that the second disc can be advanced only in the distal direction over the locking notches.

9. The medical device of claim 1, wherein the first disc comprises a flexible material, such that the first disc can be folded proximally to create a rounded distal profile.

10. The medical device of claim 1, wherein the first disc comprises a shape-memory alloy.

11. The medical device of claim 1, wherein the first and second discs comprise a shape-memory alloy, such that the discs can be folded proximally to create a rounded distal profile.

12. A delivery system for delivering a medical device for closing an anatomical aperture comprising:
a delivery tool comprising:
a delivery shaft;
an advancement member; and
a decoupling member; and
a medical device comprising:
a first disc, configured to be compressed within the delivery shaft, comprising a distal surface and a proximal surface;
a shaft attached to the first disc and extending from the proximal surface of the first disc; and
a second disc, configured to be compressed within the delivery shaft, comprising a distal surface and a proximal surface, the second disc having a hole, wherein the hole is sized such that the shaft can fit through the hole;
wherein the advancement member is configured to advance the second disc distally along the shaft, and wherein the decoupling member is configured to remove a proximal portion of the shaft.

13. The delivery system of claim 12, wherein the advancement member comprises a tube having an interior lumen sized to fit about the shaft, configured to push the second disc distally along the shaft.

14. The delivery system of claim 12, wherein the advancement member is configured to rotate the second disc along a threaded exterior surface of the shaft.

15. The delivery system of claim 12, wherein the decoupling member comprises a tube having an interior lumen sized to fit about the shaft, and at least one blade located at a distal end of the tube, configured to cut a proximal portion of the shaft.

16. A method of closing an anatomical aperture comprising:
providing a delivery system for delivering a medical device for closing an anatomical aperture, the delivery system comprising:
a delivery tool comprising:
a delivery shaft; and
an advancement member; and
a medical device comprising:
a first disc, configured to be compressed within the delivery shaft, comprising a distal surface and a proximal surface;
a shaft attached to the first disc and extending from the proximal surface of the first disc; and
a second disc, configured to be compressed within the delivery shaft, comprising a distal surface and a proximal surface, the second disc having a hole, wherein the hole is sized such that the shaft can fit through the hole;
wherein the advancement member is configured to advance the second disc distally along the shaft;
loading the medical device into the delivery shaft;
inserting the delivery system into a body lumen;
advancing the delivery system to a deployment location;
deploying the first disc;
retracting the delivery system to engage the first disc with an interior tissue wall;
advancing the second disc along the shaft to engage the second disc with an exterior tissue wall; and
retracting the delivery shaft to deploy the second disc.

17. The method of claim 16, further comprising removing the delivery system from the body lumen.

18. The method of claim 16, further comprising advancing a decoupling mechanism within the delivery shaft and removing a portion of the shaft located proximally from the second disc.

19. The method of claim 18, wherein removing the portion of the shaft comprises cutting the shaft.

20. The method of claim 16, wherein advancing the second disc along the shaft comprises pushing the second disc with the advancement member.

21. The method of claim 16, wherein advancing the second disc along the shaft comprises rotating the second disc along a threaded exterior surface of the shaft with the advancement member.

22. The method of claim 16, wherein the anatomical aperture is an opening in a heart wall.
